(19) United States
(12) Patent Application Publication
(10) Pub. No.: US 2009/0053816 A1
(43) Pub. Date: Feb. 26, 2009

(54) HEMOGLOBIN-BASED METHODS FOR PROPHYLAXIS, DIAGNOSIS AND/OR TREATMENT OF RETINAL DISORDERS

(75) Inventors: Tongalp H. Tezel, Louisville, KY (US); Henry J. Kaplan, Louisville, KY (US); Jon Klein, Louisville, KY (US)

Correspondence Address: STITES & HARBISON, PLLC, 400 W MARKET ST, SUITE 1800, LOUISVILLE, KY, 40202-3352, US

(73) Assignee: UNIVERSITY OF LOUISVILLE RESEARCH FOUNDATION, INC., Louisville, KY (US)

(21) Appl. No.: 12/294,409

(22) PCT Filed: Mar. 26, 2007
(86) PCT No.: PCT/US07/64940

§ 371 Date: Oct. 23, 2008

Related U.S. Application Data
(60) Provisional application No. 60/785,884, filed on Mar. 24, 2006.
(60) Provisional application No. 60/796,224, filed on Apr. 28, 2006.

(51) Int. Cl. G01N 33/72; C12Q 1/00; A61K 38/00; A61K 39/395; A61P 27/00; A61B 5/05; A61B 17/20

(52) U.S. Cl. 436/67; 436/66; 435/4; 514/8; 424/172.1; 600/407; 604/22

(57) ABSTRACT

The presently disclosed subject matter provides methods of diagnosing retinal disorders in subjects by measuring hemoglobin and modified hemoglobin in the subjects. The presently disclosed subject matter further provides methods of treating retinal disorders in subjects by decreasing hypoxia in retinal tissue of the subjects through modulation of hemoglobin levels and activities in the retinal tissue.

### RELATED APPLICATIONS

This application claims priority from U.S. Provisional Patent Application Ser. No. 60/785,884 filed Mar. 24, 2006 and U.S. Provisional Patent Application Ser. No. 60/796,224 filed Apr. 28, 2006, the entire disclosures of which are incorporated herein by this reference.

### GOVERNMENT INTEREST

This presently disclosed subject matter was made with U.S. Government support under Grant No. 5K08EY016120-02 awarded by the National Institutes of Health. Thus, the U.S. Government has certain rights in the presently disclosed subject matter.

### TECHNICAL FIELD

The presently disclosed subject matter relates to methods for diagnosing retinal disorders and therapeutic treatments for retinal disorders. In particular, the presently disclosed subject matter relates to diagnostic and therapeutic methods based on detection and treatment of modified hemoglobin and reduced levels of functional hemoglobin.

### BACKGROUND

The retina is the light-sensitive portion of the eye. The retina contains the cones and rods (photoreceptors), which are the photosensitive cells of the eye. The rods contain rhodopsin, the rod photopigment, and the cones contain three distinct photopigments, which respond to light and transmit signals through successive neurons to ultimately trigger a neural discharge in the output cells of the retina, the ganglion cells. The signal is carried by the optic nerve to the visual cortex where it is registered as a visual stimulus.

In the center of the retina is the macula lutea, which is about ⅓ to ½ cm in diameter. The macula provides detailed vision, particularly in the center (the fovea), because the cones are higher in density. Blood vessels, ganglion cells, inner nuclear layer and cells, and the plexiform layers are all displaced to one side (rather than resting above the cones), thereby allowing light a more direct path to the cones.

Under the retina is the choroid, a collection of blood vessels embedded within a fibrous tissue, and the pigmented epithelium (PE), which overlays the choroid layer. The choroidal blood vessels provide nutrition to the retina (particularly its visual cells). The choroid and PE are found at the posterior of the eye.

The retinal pigment epithelial (RPE) cells, which make up the PE, produce, store, and transport a variety of factors that are responsible for the normal function and survival of photoreceptors. RPE are multifunctional cells that transport metabolites to the photoreceptors from their blood supply, the Wchorio capillaris of the eye. The RPE cells also function to recycle vitamin A as it moves between the photoreceptors and the RPE during light and dark adaptation. RPE cells also function as macrophages, phagocytizing the rhythmically-shed tips of the outer segments of rods and cones, which are produced in the normal course of cell physiology. Various ions, proteins and water move between the RPE cells and the interphotoreceptor space, and these molecules ultimately effect the metabolism and viability of the photoreceptors.

Diseases or injury to the retina and supporting tissue can lead to blindness if retinal cells are injured or killed. The photoreceptor cells are particularly susceptible to injury since they are often the first cells to degenerate or suffer damage as a result of a traumatizing event or disease condition. Hereditary defects in specific photoreceptor genes, retinal detachment, circulatory disorders, overexposure to light, toxic effects to drugs, and nutritional deficiencies are among the wide array of causes that can result in the death of photoreceptor cells. Developmental and hereditary diseases of the retina account for around 20 percent of all legal blindness in the United States. See Report of the Retinal and Choroidal Panel: Vision Research—A National Plan 1983-1987, vol. 2, part 1, summary page 2. For example, retinitis pigmentosa (RP), a genetic based progressive disease is characterized by incremental loss of peripheral vision and night blindness, which is due in large part to the loss of photoreceptor cells. RP is a group of hereditary diseases and presently afflicts about one in 3000 persons worldwide. Wong, F. (1995) Arch. Opthalmol. 113: 1245-47. Total blindness is the usual outcome in more progressive stages of this disease. Macular degeneration, another major cause of blindness, is a complex group of disorders that affects the central or predominantly cone portion of the retina. Diabetic retinopathy, a frequent complication in individuals with diabetes mellitus, is estimated to be the fifth leading cause of new blindness. However, it is the second leading cause of blindness among individuals of 45-74 years of age. Moreover, these problems are only expected to get worse as the general population ages.

Various disorders of the retina and/or affecting the retina (e.g. disorders affecting the choroid and other retina supporting tissues) are amenable to prophylaxis and/or therapy as long as the prophylactic and/or therapeutic regimens are timely initiated. However, one limitation on timely intervention lies in the difficulty in detection of early stages of various pathophysiological processes. In particular, there is a relative paucity of routine diagnostic tests to be performed on peripheral tissues (i.e., other than those found in the eye) that can reveal the progress of pathophysiological processes in, or affecting, the retina. As such, there is an unmet need for additional diagnostic procedures for diagnosis of retinal disorders, especially simple tests performed on peripheral tissues, and novel therapeutic procedures for treating retinal disorders.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides in some embodiments, a method for diagnosing a retinal disorder, or the risk of developing the retinal disorder, in a subject. In some embodiments, the method comprises providing a biological sample from the subject, determining an amount of modified hemoglobin in the sample, and comparing the amount of modified hemoglobin to a control level. In other embodiments, the method comprises determining an amount of hemoglobin, modified hemoglobin, or both present in retinal tissue of the subject and comparing the amount of hemoglobin, modified hemoglobin, or both to a control level. In some embodiments, determining the amount of hemoglobin, modified hemoglobin, or both comprises non-invasive imaging of the retinal tissue to measure the amount of hemoglobin, modified hemoglobin, or both present in the retinal tissue. The subject can then be diagnosed as having the retinal disorder, or being at risk for developing the retinal disorder, if there is a measurable difference in the amount of modified hemoglobin in the sample and/or the amount of hemoglobin, modified hemoglobin, or both in the retinal tissue, as compared to the control level.

The presently disclosed subject matter further provides in some embodiments a method for determining whether to initiate or continue prophylaxis or treatment of a retinal disorder in a subject. In some embodiments, the method comprises providing a series of biological samples over a time period from the subject, analyzing the series of biological samples to determine an amount of modified hemoglobin in each of the biological samples, and comparing any measurable change in the amounts of modified hemoglobin in each of the biological samples to determine whether to initiate or continue the prophylaxis or treatment of the retinal disease. In some embodiments, the series of biological samples comprises a first biological sample collected prior to initiation of the prophylaxis or treatment for the retinal disorder and a second biological sample collected after initiation of the prophylaxis or treatment. In some embodiments, the series of biological samples comprises a first biological sample collected prior to onset of the retinal disorder and a second biological sample collected after onset of the retinal disorder.

In other embodiments of the method for determining whether to initiate or continue prophylaxis or treatment of a retinal disorder in a subject, the method comprises determining a series of amounts of hemoglobin, modified hemoglobin, or both present in retinal tissue of the subject over a time period and comparing any measurable change in the amounts of hemoglobin, modified hemoglobin, or both present in the retinal tissue of the subject over the time period to determine whether to initiate or continue the prophylaxis or therapy of the retinal disease. In some embodiments, determining the amounts of hemoglobin, modified hemoglobin, or both comprises non-invasive imaging of the retinal tissue to measure the amounts of hemoglobin, modified hemoglobin, or both present in the retinal tissue over the time period. In some embodiments, the time period begins prior to initiation of the prophylaxis or treatment for the retinal disorder and ends after initiation of the prophylaxis or treatment. In some embodiments, the time period begins prior to onset of the retinal disorder and ends after onset of the retinal disorder.

The presently disclosed subject matter still further provides in some embodiments, a method of treating a retinal disorder in a subject. In some embodiments, the method comprises decreasing hypoxia in retinal tissue of the subject by increasing an amount of hemoglobin, increasing an activity of hemoglobin, decreasing an amount of modified hemoglobin, decreasing an activity of modified hemoglobin, or combinations thereof in the retinal tissue of the subject. In some embodiments, increasing the amount of hemoglobin in the retinal tissue comprises administering a therapeutically effective amount of erythropoietin to the subject. In some embodiments, decreasing the concentration of modified hemoglobin in the retinal tissue comprises removing retinal tissue comprising the modified hemoglobin. In some embodiments, decreasing the activity of modified hemoglobin comprises administering a pharmaceutical composition comprising an anti-CD36 molecule, such as for example is an antibody or an aptamer, to the subject.

In some embodiments of the presently disclosed diagnostic and therapeutic methods, the retinal disorder is a retinal disorder selected from the group consisting of age-related macular degenerations, diabetic retinopathies, retinopathy of prematurity (ROP), ischemic vasculopathies, inherited retinal dystrophies, retinal detachment, aberrant angiogenesis, and glaucoma. Further, in some embodiments, the retinal disorder produces in the subject one or more physiological abnormalities selected from the group consisting of choroidal neovascularization (CNV), retinal angiomatous proliferation (RAP), intraretinal microvascular abnormalities, pre-retinal neovascularization, choroidal angiogenesis, and choroidal vasculopathy.

In some embodiments of the presently disclosed diagnostic and therapeutic methods, the modified hemoglobin comprises oxidized hemoglobin, s-nitrosylated hemoglobin, acetylated hemoglobin, glycated hemoglobin, or combinations thereof.

In some embodiments of the presently disclosed diagnostic and therapeutic methods, the retinal tissue comprises retinal pigment epithelial (RPE) cells, Bruch's membrane, choroid, or combinations thereof.

Accordingly, it is an object of the presently disclosed subject matter to provide hemoglobin-based methods and compositions for optimizing prophylaxis, diagnosis and/or treatment of ocular disorders, including but not limited to retinal disorders. This object is achieved in whole or in part by the presently disclosed subject matter.

An object of the presently disclosed subject matter having been stated hereinabove, and which is achieved in whole or in part by the presently disclosed subject matter, other objects and advantages will become evident to those of ordinary skill in the art after a study of the following description of the presently disclosed subject matter, drawings, and non-limiting examples.

### BRIEF DESCRIPTION OF THE DRAWINGS

FIG. 1 is a photograph of a Western blot showing hemoglobin expression levels in cultured retinal pigment epithelial (RPE) cells under normal and hypoxic oxygen conditions.

FIG. 2 is a photograph of an RT-PCR gel showing Hgb-β expression levels in cultured RPE cells under normal and hypoxic oxygen conditions.

FIG. 3 is a photograph of hemoglobin side chain spots on two dimensional gels showing hemoglobin expression levels are decreased in cultured RPE cells over time in the tissue culture environment.

FIG. 4 is a photograph of hemoglobin side chain spots on two dimensional gels showing hemoglobin expression levels are increased in a hypoxic environment (5% O₂).

FIG. 5 is a photograph of a two-dimensional protein gel of RPE cell extract showing presence and clustering of Hgb side chains (α and β).

FIG. 6 is a panel of four photographs, each of two-dimensional protein gels of RPE cell extracts from bovine (top left), mouse (top right), pig (bottom left), and rat (bottom right), each showing presence of Hgb side chains.

FIG. 7 is a photograph of a Western blot showing the presence of Hgb side chains within human RPE cells.

FIG. 8 is a photograph of an RT-PCR gel showing the expression Hgb α1, α2, γ and β chains by human RPE cells.

FIG. 9 is a panel of photomicrographs confirming the presence of Hgb within human RPE as small granular deposits located predominantly close to the basal membrane, as detected by immunohistochemistry (anti-human Hgb).

FIGS. 10A-10F are immunoelectron photomicrographs confirming the presence and localization of Hgb within RPE cells.

FIG. 11 is a panel of photographs showing age-related decline of the amount of hemoglobin in the Bruch's membrane as demonstrated by comparison of young (left panels) and old (right panels) Bruch's membrane samples.

### DETAILED DESCRIPTION

The details of one or more embodiments of the presently disclosed subject matter are set forth in the accompanying description below. Other features, objects, and advantages of the presently disclosed subject matter will be apparent from the Detailed Description, Figures, and Claims. All publications, patent applications, patents, and other references mentioned herein are incorporated by reference in their entirety. In case of conflict, the present specification, including definitions, will control.

Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

Hypoxia of the outer retina plays an important role in various retinal pathologies, including age-related macular degeneration, diabetic retinopathy, cystoid macular edema, and retinal vascular occlusions. The outer retina, being metabolically more active even than brain, kidney or heart, requires greater amounts of oxygen than many tissues. This demand can be supplied by oxygen freely diffusing from the choroidal vasculature. However, unlike retinal vasculature, choroidal vessels lack autoregulation, which predisposes outer retina to hypoxic damage. Relatively longer survival of photoreceptors in hypoxia, compared with other neurons, and different photoreceptor hypoxic damage thresholds between species suggests the presence of an alternative oxygen delivery and/or storage mechanism in the outer retina. As disclosed in detail herein, the presently disclosed subject matter provides evidence that hemoglobin carries out such a function in the outer retina.

Hemoglobin (Hgb) is conventionally known as the principal oxygen-transport protein of mammalian erythrocytes. When contained in adult erythrocytes, Hgb exists as a tetramer structure composed of two oxygen linked α-β dimers, each having a molecular weight of about 32 KDa. Each α and β subunit of each dimer has a protein chain and a heme molecule. The sequences of the α and β protein chains are known. The Hgb molecule is unique among other oxygen-binding globins due to its quaternary structure that allows allosteric regulation of its oxygen-carrying capacity, so that once oxygen binds to one of the Hgb subunits it creates a homotopic affinity increase of other subunits to bind oxygen. However, in metabolically active tissues, such as in outer retina, unloading of oxygen from a Hgb subunit decreases the binding affinity of oxygen to the remaining subunits and facilitates maximal oxygen delivery. Hgb molecules can also bind CO₂ to its N-amino terminal as a carbamate and clears ˜15% of the CO₂ from the tissue. Recent studies have shown that Hgb can be expressed in various non-erythroid tissues as well. See, e.g., Newton D A et al, J Biol Chem (2006); see also Liu L et al, Proc Natl Acad Sci USA (1999).

Similarly, myoglobin is known as the principal oxygen-transport protein of certain peripheral tissues such as skeletal muscle. However, the role of various non-hemoglobin oxygen-transport proteins in vertebrate tissues has recently been shown to be surprisingly complex, with the demonstration of the inducibility of myoglobin in non-muscle tissue and the discovery and characterization of distinct oxygen-transport proteins such as neuroglobin and cytoglobin. See, e.g., Fraser J et al, Hypoxia-inducible myoglobin expression in nonmuscle tissues, Proc Natl Acad Sci USA (2006); see also Ostojic J et al, Neuroglobin and cytoglobin: oxygen-binding proteins in retinal neurons, Invest Opthalmol V is Sci (2006).

The presently disclosed subject matter provides, for the first time, evidence of hemoglobin expression in human retinal pigment epithelium cells (RPE). Accordingly, the present subject matter provides hemoglobin-based methods for optimizing prophylaxis, diagnosis, and/or treatment of retinal disorders.

“Retinal disorders”, as the term is used herein, refers to any changes to the retinal tissue of the eye that can affect viability and/or functioning of the retina, cells therein, and supporting tissue, including but not limited to changes in vision. Exemplary retinal disorders include, but are not limited to: age-related macular degeneration, such as for example non-exudative and exudative forms of age-related macular degeneration; diabetic retinopathy, such as for example non-proliferative and proliferative forms of diabetic retinopathy; retinopathy of prematurity (ROP); ischemic vasculopathies, such as for example central retinal vein occlusion, branch retinal vein occlusion, central retinal artery occlusion, branch retinal artery occlusion, and coagulopathies (e.g. lupus anti-coagulant disease), vasculitis of various etiologies (both infectious and non-infectious), hemoglobinopathies (e.g. SS disease, SC disease, S-thalassemia disease), and blood dyscrasias; inherited retinal dystrophies, such as for example retinitis pigmentosa, Best's disease, and Stargardt's disease; retinal detachment, including but not limited to rhegmatogenous, traction, or combined; aberrant angiogenesis, such as for example high myopia, presumed ocular histoplasmosis, toxoplasmosis, other uveitides, trauma, and angioid streaks; and glaucoma, such as for example glaucoma secondary to abnormal O₂ transport from the RPE to the neural retina.

“Retinal tissue”, as the term is used herein, refers in part to cells and supporting biological structures of the retina as is generally understood in the art. For example, as is generally understood, the retina comprises photoreceptors, macula lutea, fovea, ganglion cells, retinal capillaries, RPE, and other structures and cells. However, “retinal tissue” as used herein is also inclusive of structures and cells of the eye that are physically associated with the retina and/or support retinal functions or survival, even though not traditionally considered part of the retina. For example, “retinal tissue” as used herein is further inclusive of choroid and Bruch's membrane.

The presently disclosed subject matter provides in some embodiments methods for diagnosing a retinal disorder, or a risk of developing the retinal disorder, in a subject. In some embodiments, the methods comprise providing a biological sample from the subject, determining an amount of modified hemoglobin in the sample, and comparing the amount of modified hemoglobin to a control level. In other embodiments, the methods comprise determining an amount of hemoglobin, modified hemoglobin, or both present in retinal tissue of the subject (e.g., RPE cells, Bruch's membrane, choroid, or combinations thereof); and comparing the amount of hemoglobin, modified hemoglobin, or both to a control level. The subject is then diagnosed as having the retinal disorder, or being at risk for developing the retinal disorder if not already suffering from the disorder, if there is a measurable difference in the amount of hemoglobin and/or modified hemoglobin in the sample and/or in the retinal tissue, as compared to the control level.

The “amount” of hemoglobin and/or modified hemoglobin determined refers to a qualitative (e.g., present or not in the measured sample), quantitative (e.g., how much is present), or both measurement of hemoglobin and/or modified hemoglobin. The “control level” is an amount (including the qualitative presence or absence) of hemoglobin and/or modified hemoglobin found in the biological sample or retinal tissue in subjects free of retinal disorders. As one non-limiting example of calculating the control level, the amount of hemoglobin and/or modified hemoglobin present in normal retinal tissues or cells (e.g., RPE cells) can be calculated and extrapolated for whole subjects. For example, in some non-limiting embodiments, each RPE cell in a human subject has been calculated to contain on average about 8.3 pg of Hgb. Further, in some embodiments, each RPE cell on average calculated to contain less than about 5 pg Hgb exhibits disrupted oxygen transport. Thus, in some embodiments, a control level for Hgb can be greater than 5 pg per cell, extrapolated to the tissue, sample or organism level, as desired.

The term “modified hemoglobin”, as used herein, refers to hemoglobin which has been altered either chemically or genetically, including for example, post-translational modifications (regarding post-translational peptide modification see e.g., Bunn H F, Haematologia (Budap), 17(2):179-86 (1984); and Harding J J, Adv Protein Chem, 37:247-334 (1985), each of which is incorporated herein by reference), and which in some instances does not function to carry and/or deliver oxygen as effectively as unmodified hemoglobin. Modified hemoglobin is known in the art to be a marker for certain pathophysiological processes. In particular, certain chemical modifications of hemoglobin can be useful as indicators of certain disease states. See, e.g., Bry L et al, Clin Chem (2001); see also Schillinger M et al, Circulation (2003); S\acute{ø}orensen M et al, Environ Health Perspect (2003); Cerami A, Prog Clin Biol Res., 195:79-90 (1985). Exemplary forms of modified hemoglobin include but are not limited to oxidized hemoglobin; s-nitrosylated hemoglobin (see Broillet M C, Cell Mol Life Sci, 55(8-9):1036-42 (1999), incorporated herein by reference); acetylated hemoglobin (see Kasten-Jolly J and Abraham E C, Biochim Biophys Acta, 27; 913(1):89-91 (1987), and Beyerbach A et al, Carcinogenesis, 27(8):1600-6 (2006), each of which is incorporated herein by reference); and glycated hemoglobin (see e.g., Winterhalter K H, Prog Clin Biol Res.; 195:109-22 (1985) regarding non-enzymatic glycosylation of proteins generally, incorporated herein by reference). For example, advanced glycation end products (AGE) are one form of glycated hemoglobin. AGE can be found in diabetic and aged eyes. Increased glucose can glycooxidize Hgb and deteriorate its function. See e.g., Nagai R et al, Ann N Y Acad. Sci., 1043:146-50 (2005); Cerami A, Prog Clin Biol Res, 195:79-90 (1985). Additional examples of modified hemoglobins include carboxyethylpyrrole (CEP) adducts derived from oxidative fragmentation of docosahexaenoate-containing lipids, which are abundant in the retina. See e.g., Quteba E et al, Proc Natl Acad Sci USA 2006 Sep. 5; 103(36):13480-4. See also, Niketic V et al, Biochem Biophys Res Commun, 239(2):435-8 (1997), which discloses mechanisms of inositol-mediated modification of hemoglobin, incorporated herein by reference. Further, oxidized hemoglobin can induce macrophages to attack and destroy RPE cells. See e.g., Amiconi G et al, Adv Exp Med Biol, 307:185-90 (1991).

In some embodiments, the retinal disorder can be a retinal disorder including but not limited to age-related macular degenerations, diabetic retinopathies, retinopathy of prematurity (ROP), ischemic vasculopathies, inherited retinal dystrophies, retinal detachment, aberrant angiogenesis, and glaucoma. Further, the retinal disorder can produce in the subject one or more physiological abnormalities, such as for example choroidal neovascularization (CNV), retinal angiomatous proliferation (RAP), intraretinal microvascular abnormalities, pre-retinal neovascularization, choroidal angiogenesis, and choroidal vasculopathy. These and other physical abnormalities can be measured to confirm diagnosis and/or monitor progress of treatment once diagnosis is made.

In some embodiments wherein modified hemoglobin is measured in a biological sample from the subject, the biological sample is one easily acquired from the subject, such as for example blood, plasma, serum, and ocular fluids (e.g., vitreous and aqueous ocular fluids). The modified hemoglobin in the biologic sample can then be measured utilizing any of a number of methodologies generally known in the art. See for example, Rodkey F L et al, Clin. Chem. (1979), incorporated herein by reference, which teaches spectrophotometric measurement of methemoglobin (oxidized hemoglobin) in blood. See also, U.S. Pat. No. 4,268,270, incorporated herein by reference, which teaches a process and apparatus for measuring glycosylated hemoglobin by assaying bound glyco groups on the hemoglobin. See also, Miksik I and Deyl Z, J Chromatogr B Biomed Sci Appl. 699 (1-2):311-45 (1997), incorporated herein by reference, which discloses methods for separation of selected protein species after glycation and other carbonyl-mediated modifications. See also, Marzocchi B et al, Pediatr Res. October; 58(4):660-5 (2005), incorporated herein by reference, which discloses hypoxia-induced modifications to proteins and detection methods.

In some embodiments where an amount of hemoglobin and/or modified hemoglobin is measured in retinal tissue, non-invasive imaging of the retinal tissue can be utilized to measure the amount of hemoglobin and/or modified hemoglobin present in the retinal tissue. Additionally, in some embodiments, hemoglobin and/or modified hemoglobin in retinal tissue can be measured indirectly through the measurement of hemoglobin degradation products as an indicator of retinal disease or predilection therefore. See e.g., Ahmed N et al, Diabetologia, 48(8):1590-603 (2005), incorporated herein by reference, which discloses various degradation products of proteins damaged by glycation, oxidation and nitration in clinical type 1 diabetes. For example, iron (Fe³⁺) deposition in retinal tissue can be measured as a hemoglobin degradation product and potential indicator of retinal disease.

Non-invasive imaging techniques of retinal tissue applicable to measuring hemoglobin, modified hemoglobin, and/or degradation products thereof are generally known in the art. Any such suitable generally known techniques are intended to be encompassed within the presently disclosed subject matter. Exemplary non-limiting, non-invasive imaging techniques suitable for use with the presently disclosed methods include non-invasive imaging of the RPE, Bruch's membrane, and/or choroid for modified Hgb using, for example, scanning laser opthalmoscopy (SLO; see Webb, R. H. et al, Applied Optics, 26, 1492-1499 (1987)) using a scanning laser opthalmoscope (e.g., Rodenstock Instruments, Munich, Germany); adaptive optics; positron emission tomography (PET); optical coherence tomography (OCT; see Huang D. et al., Science 254, pp. 1178-1181 (1991), and Fercher A. F., J. Biomed. Opt., 1(2): pp. 157-173 (1996)); dual photon microscopy; fluorescence microscopy; photoacoustic imaging (Kolkman et al, Lasers Med Sci, 21 (3):134-9 (2006), and Wang et al, J Biomed Opt, 11(2):024015 (2006)); near-infrared (NIR) imaging (Srinivasan S et al, Acad Radiol, 13(2):195-202 (2006)); hyperspectral imaging (Sorg B S et al, J Biomed Opt, 10(4):44004 (2005)); fundus spectrometry (Schweitzer D et al, Klin Monatsbl Augenheilkd, 222(5):396-408 (2005)); and optical tomography (Zhu Q et al, Neoplasia, 7(3):263-70 (2005).

In addition to directly or indirectly measuring hemoglobin and/or modified hemoglobin in the subject for diagnosing or predicting retinal disease, molecular markers of RPE or photoreceptor cell death or hypoxia can be measured to confirm or further facilitate diagnosis. For example, genes expressed by dysfunctional and/or dying RPE cells can be determined as an indicator of retinal disease, which include but are not limited to CD36, HLA-DR, CD68, vitronectin, apolipoprotein E, clusterin and S-100. The dysfunctional and/or dying RPE-associated markers can be detected on the molecular level, e.g. by detecting the identity, level, and/or activity of the gene, mRNA transcript, or encoded protein, as is generally known in the art.

In view of the above guidance provided for the diagnostic methods disclosed herein, following are a number of prophetic examples of utilization of the diagnostic methods. The examples are intended only to provide guidance as to application of the methods and are not intended to limit the scope of the presently disclosed subject matter in any way.

In a first prophetic example, a biological sample (e.g., peripheral blood) is collected from each of a number of individuals. The amount (e.g., relative abundance) of glycated hemoglobin in each sample is noted. Through use of one or methods disclosed herein or otherwise known in the art, the presence or absence in each individual of any one or more of the retinal disorders disclosed herein is also noted. It is found that the relative abundance of glycated hemoglobin is positively correlated with the presence of one or more of the listed abnormalities.

In another prophetic example, a biological sample (e.g., peripheral blood) is collected from each of a number of individuals. The amount (e.g., relative abundance) of oxidized hemoglobin in each sample is noted. Through use of one or methods disclosed herein or otherwise known in the art, the presence or absence in each individual of any one or more of the retinal disorders disclosed herein is also noted. It is found that the relative abundance of oxidized hemoglobin is positively correlated with the presence of one or more of the listed abnormalities.

In still another prophetic example, a standard set of retinal examinations known in the art is performed on each of a number of individuals. A sample of peripheral blood is collected from each of these individuals as well. It is found that the median amount (e.g., relative abundance) of modified hemoglobin (e.g., glycated and/or oxidized hemoglobin) is greater in samples taken from individuals in whom any of the retinal disorders disclosed herein is noted than the median relative abundance of glycated and/or oxidized Hgb in samples taken from individuals in whom any of retinal disorders disclosed herein is not noted.

The presently disclosed subject matter further provides in some embodiments methods for determining whether to initiate or continue prophylaxis or treatment of a retinal disorder in a subject. In some embodiments, the methods comprise providing a series of biological samples over a time period. In other embodiments, the methods comprise determining a series of amounts of hemoglobin, modified hemoglobin, or both present in retinal tissue of the subject over a time period. Next, the methods comprise comparing any measurable change in the amounts of hemoglobin and/or modified hemoglobin in each of the biological samples and/or present in the retinal tissue over the time period to determine whether to initiate or continue the prophylaxis or treatment of the retinal disease. A measurable change in the amount of hemoglobin and/or modified hemoglobin would be indicative of the retinal disease and would result in initiation or continuation of treatment of the subject for the retinal disease. The time period and frequency of sampling within the time period can vary according to need. For example, for elderly human subjects not otherwise exhibiting symptoms of a retinal disorder, it may be desirable to provide biological samples for testing annually. However, if symptoms of a retinal disorder are present, or the subject has risk factors (e.g., drusen, family history, obesity, smoking, etc.), it can be desirable to increase the frequency of sampling and testing. Further, if treatment for a retinal disorder has been initiated, the frequency of testing can be increased still further, depending on the dose and efficacy of the treatments.

In some embodiments, one or more of the series of biological samples are collected prior to initiation of the prophylaxis or treatment for the retinal disorder. One or more additional samples are then collected after the subject has been treated for a desired period of time. In this way, the samples from before and after initiation of treatment can be compared with a difference in amounts of modified hemoglobin indicative of treatment progress. For example, a decrease in the relative amounts of modified hemoglobin found in samples after initiation of treatment would be indicative of an effective treatment, and vice versa. Likewise with regard to measuring hemoglobin and/or modified hemoglobin in the retinal tissue of the subject, the time period for measuring amounts of hemoglobin or modified hemoglobin in the retinal tissue can be initiated prior to treatment and continued through treatment with measurements taken before and during treatment. Again, the before treatment measurements can be compared to the during treatment measurements to determine efficacy of the treatment. For example, a decrease in modified hemoglobin in the retinal tissue after treatment would indicate the treatment was having a positive effect on the disorder. Similarly, an increase in functioning hemoglobin would likely indicate the treatment was having a positive effect, depending on the treatment chosen.

In some embodiments, the retinal disorder can be a retinal disorder including but not limited to age-related macular degenerations, diabetic retinopathies, retinopathy of prematurity (ROP), ischemic vasculopathies, inherited retinal dystrophies, retinal detachment, aberrant angiogenesis, and glaucoma. Further, the retinal disorder can produce in the subject one or more physiological abnormalities, such as for example choroidal neovascularization (CNV), retinal angiomatous proliferation (RAP), intraretinal microvascular abnormalities, pre-retinal neovascularization, choroidal angiogenesis, and choroidal vasculopathy. These and other physical abnormalities can be measured to confirm diagnosis and/or monitor progress of treatment once diagnosis is made.

In some embodiments wherein modified hemoglobin is measured in a biological sample from the subject, the biological sample is one easily acquired from the subject, such as for example blood, plasma, serum, or ocular fluids. The modified hemoglobin in the biological sample can then be measured utilizing any of a number of methodologies generally known in the art, as disclosed hereinabove.

In some embodiments where an amount of hemoglobin and/or modified hemoglobin is measured in retinal tissue, non-invasive imaging of the retinal tissue can be utilized to measure the amount of hemoglobin and/or modified hemoglobin present in the retinal tissue, as disclosed in detail hereinabove. Additionally, in some embodiments, hemoglobin and/or modified hemoglobin in retinal tissue can be measured indirectly through the measurement of hemoglobin degradation products as an indicator of retinal disease or predilection therefore. For example, iron (Fe³⁺) deposition in retinal tissue can be measured as a hemoglobin degradation product and potential indicator of retinal disease.

In addition to directly or indirectly measuring hemoglobin and/or modified hemoglobin in the subject for determining whether to initiate or continue prophylaxis or treatment of a retinal disorder, molecular markers of RPE or photoreceptor cell death or hypoxia can be measured to confirm or further facilitate diagnosis. For example, genes expressed by dysfunctional and/or dying RPE cells can be determined as an indicator of retinal disease, which include but are not limited to CD36, HLA-DR, CD68, vitronectin, apolipoprotein E, clusterin, and S-100. The dysfunctional and/or dying RPE-associated markers can be detected on the molecular level as disclosed hereinabove.

In view of the above guidance provided for the methods for determining whether to initiate or continue prophylaxis or treatment of a retinal disorder disclosed herein, following is a prophetic example for utilization of the methods. The example is intended only to provide guidance as to application of the methods and is not intended to limit the scope of the presently disclosed subject matter in any way.

In the present prophetic example, one or more regimes of prophylaxis and/or therapy known in the art to be useful for the prophylaxis and/or therapy of retinal disease or disclosed herein are initiated in accordance with the finding that a subject has presented with an elevated level of modified hemoglobin (e.g., oxidized and/or glycated Hgb) in a biological sample or as measured in a retinal tissue, or a decreased measurement of functional hemoglobin in the retinal tissue, but has not yet presented with overt and/or readily-detected retinal diseases and/or associated physiological abnormalities upon a conventional retinal examination. In this way, the progress of the disease or abnormality in question is forestalled or slowed, leading to an improved long-term clinical outcome.

The presently disclosed subject matter further provides methods of treating a retinal disorder in a subject. As previously disclosed, hypoxia of the retina plays a role in retinal disorders. Further, the presently disclosed subject matter discloses, for the first time, that tissues of the retina express hemoglobin and hemoglobin expression is upregulated in these tissues under hypoxic conditions. Thus, manipulation of functional hemoglobin within retinal tissues can reduce hypoxia in retinal tissues, thereby treating the retinal disorder and associated physiological abnormalities. As such, in some embodiments, the treatment methods comprise decreasing hypoxia in retinal tissue of a subject by increasing an amount of hemoglobin, increasing an activity of hemoglobin, decreasing an amount of modified hemoglobin (e.g., oxidized and/or glycated hemoglobin), decreasing an activity of modified hemoglobin, or combinations thereof in the retinal tissue of the subject.

In some embodiments, the retinal disorder treated can be a retinal disorder including, but not limited to age-related macular degenerations, diabetic retinopathies, retinopathy of prematurity (ROP), ischemic vasculopathies, inherited retinal dystrophies, retinal detachment, aberrant angiogenesis, and glaucoma. Further, the retinal disorder can produce in the subject one or more physiological abnormalities, such as for example choroidal neovascularization (CNV), retinal angiomatous proliferation (RAP), intraretinal microvascular abnormalities, pre-retinal neovascularization, choroidal angiogenesis, and choroidal vasculopathy, which can also be treated by the therapeutic methods. In some embodiments, the diagnostic methods disclosed herein can be used in combination with the therapeutic methods such that treatment can be initiated prior to manifestation of symptoms or effects detectable using traditional diagnostic tests for the retinal disorder and/or associated physiological abnormalities. This approach can help to prevent or reduce damage to retinal tissue (and vision), which can in some instances be irreversible, otherwise occurring when the retinal disorder is treated at a later stage of progression.

In some embodiments of the treatment methods, the retinal tissue (e.g., RPE cells, Bruch's membrane, choroid, or combinations thereof) is treated directly (e.g., with a laser), to thereby reduce hypoxic conditions in the tissue. In some embodiments, the retinal is treated by administration of a therapeutic agent systemically or to the eye, which then targets the retinal tissue directly, or indirectly by direct interaction with an intermediary molecule or cell that then directly or indirectly acts on the retinal tissue, to thereby reduce hypoxic conditions in the tissue. Exemplary, non-limiting methods for decreasing hypoxia in retinal tissue by increasing an amount of hemoglobin, increasing an activity of hemoglobin, decreasing an amount of modified hemoglobin, decreasing an activity of modified hemoglobin, or combinations thereof in the retinal tissue of a subject are now provided.

In some embodiments, hypoxia in retinal tissue of a subject can be reduced by increasing an amount and/or activity (e.g., oxygen-carrying/delivery capacity) of functional hemoglobin in the retinal tissue by correction of relative hypoxia (e.g., anemia) in the subject as a whole. This therapeutic approach can be particularly useful in subjects who have been diagnosed as at risk for developing retinal disorders using the presently disclosed novel diagnostic methods alone, or in combination with known techniques for assessing retinal disorder risk. For example, subjects at an increased risk for aberrant angiogenesis, particularly due to systemic or retinal-specific Hgb abnormalities, can be administered therapeutic compositions that reduce the systemic or local hypoxia. For example, therapeutics for treating anemia can be administered, which can thereby result in an increase in functioning hemoglobin in the retinal tissue. Therapeutic molecules for treating anemia are known in the art and include, for example, erythropoietin, synthetic oxygen carriers, encapsulated transfected-cell transplants, intraocular long-acting slow-releasing Hgb implants, gene treatment with plasmids or viral vectors, and cell targeted nano-particles loaded with Hgb. Thus, in some embodiments, methods of treating or preventing a retinal disorder comprise administering to the subject a therapeutically effective amount of a compound that increases amounts of functioning hemoglobin in retinal tissue (e.g., erythropoietin, etc.) of the subject.

In addition, genetic modification of hemoglobin-producing cells (e.g., RPE cells) using gene therapy techniques generally known in the art can be utilized to increase functioning hemoglobin and reduce hypoxia in the retinal tissue. In general, gene therapy comprises exposing the subject to a gene therapy vector encoding a gene of interest (e.g., functioning hemoglobin) or encoding a nucleotide sequence that influences expression of a gene of interest. The therapy vector can be carried to cells of the subject by any of numerous vehicles known in the art, including but not limited to polyamines and neutral polymers (synthetic or nonsynthetic, such as gelatin); nanoparticles (see e.g., Vijayanathan V et al, Biochemistry, 41(48):14085-94 (2002) and Brannon-Peppas L & Blanchette J O, Adv Drug Deliv Rev 56(11):1649-59 (2004), incorporated herein by reference); liposomes (see e.g., Felnerova D et al, Curr Opin Biotechnol 15(6):518-29 (2004), incorporated herein by reference); and viral vectors such as for example retrovirus, herpes virus, and parvovirus (see generally e.g., U.S. Pat. Nos. 6,458,587; 6,489,162; 6,491,907; and 6,548,286, incorporated herein by reference). Exemplary gene therapy methods are described in U.S. Pat. Nos. 7,105,346; 7,052,881; 7,022,319; 7,018,826; 5,279,833; 5,286,634; 5,399,346; 5,646,008; 5,651,964; 5,641,484; and 5,643,567, the contents of each of which are herein incorporated by reference.

In some embodiments, hypoxia in retinal tissue of a subject can be reduced by decreasing amounts of modified hemoglobin in retinal tissue of the subject. For example, in some embodiments, retinal tissue comprising modified hemoglobin can be removed from the retina and/or surrounding tissue (e.g., choroid) using techniques for tissue debridement known in the art. For example, laser therapy, including photodynamic therapy, can be used to debride RPE and choroidal cells comprising modified Hgb. Photodynamic therapy (PDT) is a treatment that uses a compound, called a photosensitizer or photosensitizing agent, and a particular type of light to kill cells. The photo sensitizer is administered either to the tissue to be treated or systemically, where it then localizes to the tissue to be treated. When photosensitizers are exposed to a specific wavelength of light, they produce toxic species that kills nearby cells. See for example, Vrouenraets M B et al, Anticancer Research, 23:505-522 (2003), incorporated herein by reference. In some embodiments, reducing degradation products of hemoglobin and/or modified hemoglobin can be utilized as well to reduce hypoxia in retinal tissue. For example, chelation of iron present in retinal tissue can reduce hypoxia in the retinal tissue.

In some embodiments of the therapeutic methods, hypoxia in retinal tissue of a subject can be reduced by decreasing detrimental activity of modified hemoglobin in retinal tissue of the subject. For example, the effect of modified Hgb on retinal cell function, including cell death, can be blocked. As one non-limiting example, neutralizing cell membrane molecules, such as CD36 that serves as a macrophage ligand, with antibodies or aptamers can reduce hypoxia in retinal tissue resulting from modified hemoglobin effects. Thus, in some embodiments, decreasing the activity of modified hemoglobin comprises administering a pharmaceutical composition comprising an anti-CD36 molecule, such as for example an antibody (e.g., monoclonal anti-human CD36 antibodies available from R&D Systems, Minneapolis, Minn., U.S.A.) or an aptamer, to the subject. As another non-limiting example, the quenching of modified Hgb can be reversed and thereby reduce hypoxia in the retinal tissue. For example, free radical scavengers, such as superoxide dismutase (e.g., catalase) can be administered systemically or locally to the subject. Further, in some embodiments, increasing the clearance of modified hemoglobin degradation products, such as for example hemosiderin, can reduce hypoxia in the retinal tissue. Methodology for clearing modified hemoglobin degradation products in tissue are known in the art, and include a variety of cell therapies, pharmaceutical therapies, and photochemical treatments

Therapeutics disclosed herein for use with the presently disclosed methods can be formulated in a conventional manner using one or more physiologically acceptable carriers or excipients. Thus, the therapeutic agents described above can be formulated for administration by, for example, eye drops, injection, inhalation or insufflation (either through the mouth or the nose) or oral, buccal, parenteral or rectal administration. Treatment can also follow guidance provided in the art. For example, pulmonary administration of soluble complement receptor-1 (sCR1) to treat certain medical conditions (U.S. Pat. No. 6,169,068), intraocular administration of drugs to treat macular degeneration (U.S. Pat. No. 5,632,984), and treatment of macular edema with topical administration of carbonic anhydrase inhibitors to the eye (U.S. Pat. No. 6,046,223) have all been described in the art.

The therapeutic agents disclosed herein can be formulated for a variety of modes of administration, including systemic and topical or localized administration. Techniques and formulations generally can be found in Remmington's Pharmaceutical Sciences, Meade Publishing Co., Easton, Pa. The compositions are formulated as sterile, substantially isotonic and in full compliance with all Good Manufacturing Practice (GMP) regulations of the U.S. Food and Drug Administration. For ocular administration, a preferred technique utilizes an eye drop. For systemic administration, injection is preferred, including intramuscular, intravenous, intraperitoneal, and subcutaneous.

Exemplary methods of administration include, e.g., choroidal injection, transscleral injection or placing a scleral patch, selective arterial catheterization, intraocular administration including transretinal, subconjunctival bulbar, scleral pocket and scleral cutdown injections. An agent can also be alternatively administered intravascularly, such as intravenously (IV) or intraarterially.

In choroidal injection and scleral patching, the clinician uses a local approach to the eye after initiation of appropriate anesthesia, including painkillers and opthalmoplegics. A needle containing the therapeutic compound is directed into the subject's choroid or sclera and inserted under sterile conditions. When the needle is properly positioned the compound is injected into either or both of the choroid or sclera. When using either of these methods, the clinician can choose a sustained release or longer acting formulation. Thus, the procedure can be repeated only every several months or several years, depending on the subject's tolerance of the treatment and response.

For injection, the compounds of the invention can be formulated in liquid solutions, preferably in physiologically compatible buffers such as Hank's solution or Ringer's solution. In addition, the compounds can be formulated in solid form and redissolved or suspended immediately prior to use. Lyophilized forms are also included.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Therapeutic compositions can be administered alone or in combination with other molecules known to have a beneficial effect on damaged retinal tissue, including molecules capable of tissue repair and regeneration and/or inhibiting inflammation. Examples of useful cofactors include basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor 11, prostaglandin E2, 30 kDa survival factor, taurine, and vitamin A. (LaVail et al. (1998), Invest. Opthalmol. Vis. Sci. 39:592-602). Other useful cofactors include symptom-alleviating cofactors, including antiseptics, antibiotics, antiviral and antifungal agents and analgesics and anesthetics.

A therapeutic also can be associated with means for targeting the therapeutics to a desired tissue. For example, in some methods, a therapeutic agent can be directed to the RPE. Useful targeting molecules can be designed, for example, using the simple chain binding site technology disclosed, e.g., in U.S. Pat. No. 5,091,513. Thus, by targeted delivery, therapeutic agents are aimed to prevention or alleviation of damages to retinal tissue directly.

In clinical settings, a gene delivery system for a gene therapeutic as disclosed hereinabove can be introduced into a subject by any of a number of methods. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter, See U.S. Pat. No. 5,328,470, or by stereotactic injection, Chen et al. (1994), Proc. Natl. Acad. Sci., USA 91: 3054-3057. A sequence homologous thereto can be delivered in a gene therapy construct by electroporation using techniques described, e.g., Dev et al. (1994), Cancer Treat. Rev. 20:105-115.

The pharmaceutical preparation of the gene therapy construct or compound of the presently disclosed subject matter can comprise the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

With regard to dosages of therapeutic compositions disclosed herein, toxicity and therapeutic efficacy of such compounds can be determined by standard pharmaceutical procedures in cell cultures or experimental animals, e.g., for determining the Ld₅₀ (the dose lethal to 50% of the population) and the Ed₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the therapeutic methods of the presently disclosed subject matter, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Further with respect to the diagnostic and therapeutic methods of the presently disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis and treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

Subjects amenable to treatment include those who are presently asymptomatic but who are at risk of developing a symptomatic retinal disorder at a later time. For example, human individuals include those having relatives who have experienced such a disease, and those whose risk is determined by analysis of genetic or biochemical markers, by biochemical methods, or by other assays, including the novel diagnostic methods disclosed herein.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes 1 and 11, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Ambient Oxygen Concentration Modulates Hemoglobin (HGB) Synthesis and Release from Human Retinal Pigment Epithelium (RPE)

The present Example provides data determining the modulation of Hgb synthesis and release from human RPE by ambient oxygen concentration.

Methods

12 human donor eyes (46-66 years old) were obtained from an eye bank within 24 hours of death. After removal of the anterior segment and sensory retina, eye cups were rinsed three times with PBS and the suprachoroidal space was sealed with cyanoacrylate glue. Eye cups were then filled with 2 ml of culture medium and kept in a hypoxic (5% O₂) chamber for 16 hours. Experimentally-matched eyes were maintained in normoxic conditions (21% O₂) as controls. At the end of the incubation period, supernatants were collected and the amount of Hgb released from the RPE was determined using Hgb-ELISA. Cell count and viability of the remaining RPE were determined before mRNA and protein extraction for RT-PCR and Western blot analysis. Results were corrected to viable RPE count and Hgb content in control eye cups kept under identical conditions without RPE cells. Immunoelectron microscopy for human Hgb was used to confirm the results.

Results

No significant change was observed in RPE viability under hypoxic conditions within the experimental period. Each human RPE cell contains 0.4±0.02 pg of Hgb and releases it to the extracellular space at a rate of 0.002 pg/hr. Exposure of the experimental eyes to hypoxic conditions resulted in upregulation of Hgb expression in RPE cells and resulted in a 2.7±1.0-fold increase in the amount of released Hgb. Immunoelectron microscopy revealed clusters of Hgb within cytoplasmic vesicules. The primary exocytosis site was the RPE basal membrane, resulting in deposition of Hgb within Bruch's membrane.

Conclusions

Human RPE can actively modulate Hgb expression depending on the ambient oxygen concentration. Data showing polarized release of Hgb towards Bruch's membrane provides support for the role of Hgb as a reservoir and/or natural transporter of oxygen to the outer retina. Without wishing to be bound by theory, defects in this mechanism can cause outer retinal hypoxia, which plays a role in several pathologies, including for example age-related macular degeneration.

### Example 2

### Topographical and Age-Related Alterations in Human Bruch's Membrane Proteome

The present Example provides data defining and analyzing the topographical and involutional changes of the human Bruch's membrane proteome.

Methods

6.5 mm circular explants of human Bruch's membrane were harvested from the macula and periphery of 6 young (<50) and old (>65) donor eyes using an excimer laser. Proteins were extracted using an optimized extraction protocol and separated using two-dimensional gel electrophoresis. The differential protein expression was determined by software analysis, subject to in-gel digestion, and identified by Matrix-assisted laser desorption/ionization time-of-flight mass spectrometry (MALDI-TOF-MS) and electrospray ionization-quadruple time-of-flight MS/MS (ESI-Q-TOF MS/MS). Results were confirmed with Western blotting analysis.

Results

279 different proteins were identified in human Bruch's membrane. 98 of the proteins were common in all samples; however 16 proteins were specific to macula. Macular samples revealed higher expression of heat shock proteins 90 and 86, as well as antioxidant proteins, such as tripeptidyl peptidase, antioxidant protein-2 and superoxide dismutase, among others. Aging was associated with the appearance of multiple complement factors (C1q, C3, C7, C8 and Factor H) and fibroblast growth factor in both peripheral and macular Bruch's membrane. Aged macular Bruch's membrane revealed significant decrease in hemoglobin beta chain.

Conclusions

Human Bruch's membrane proteome shows topographical and age-related alterations reflecting cellular events in respective locations. Qualitative and quantitative changes in Bruch's membrane proteome may initiate and/or augment the cellular pathologies leading to macular degeneration.

### Example 3

### Expression of Hemoglobin in Cultured Human RPE Cells

The present Example provides data showing expression of Hgb in human RPE and its possible role in oxygen transport to the outer retina.

Methods

Primary and serially cultured RPE cells from three different human cell lines were kept in normoxic (21% O₂) and hypoxic (5% O₂) conditions at 37° C. for 16 hours. 30 μg of RPE protein were extracted and focused using pH 3-10 7.7 cm isoelectric focusing (IEF) strips. Proteins were further separated in the second dimension on 4-12% Bis-Tris gels. After SYPRO® Ruby (Molecular Probes, Eugene, Oreg., U.S.A.), staining spots of interest were cut and peptide analysis was done using tandem mass spectrometry (LC/MS/MS). Western blotting and real-time PCR were used to confirm and analyze the changes in Hgb, vascular endothelial growth factor (VEGF), and hypoxia-inducible factor 1, alpha subunit (HIF1-α) expression. Quantification of protein expression at different oxygen levels was done using PHORETIX 2D EXPRESSION® software (Shimadzu Biotech, Kyoto, Japan).

Results

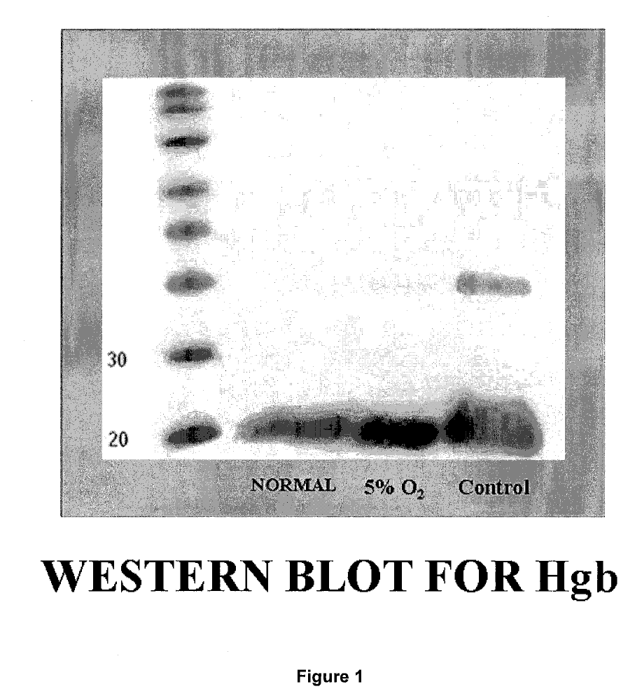

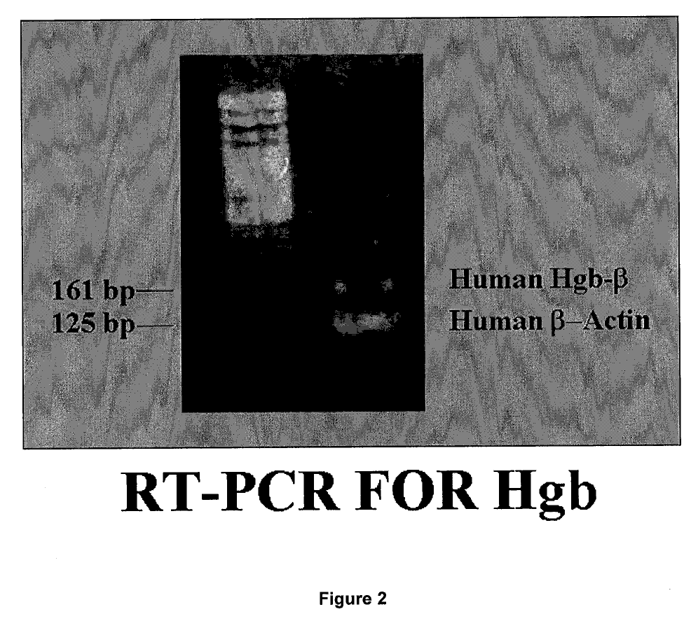

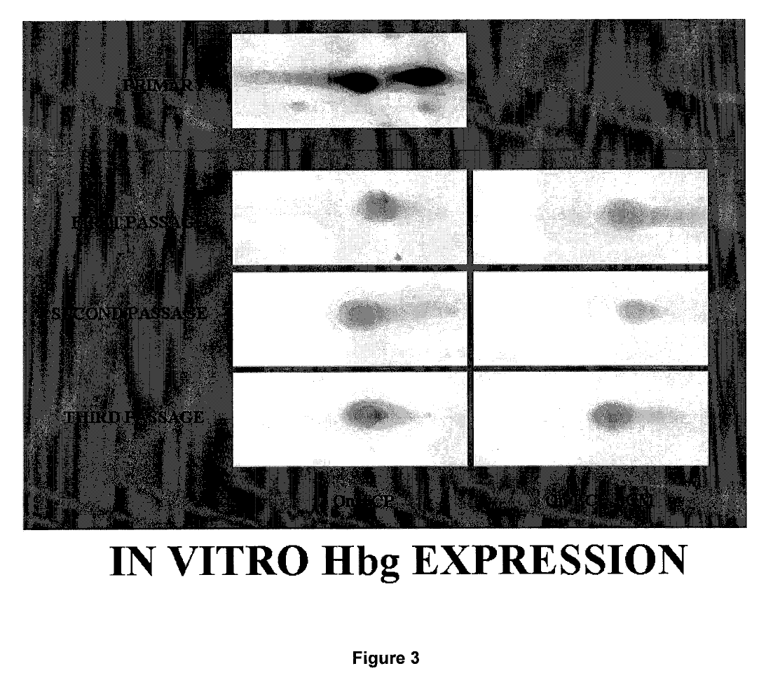

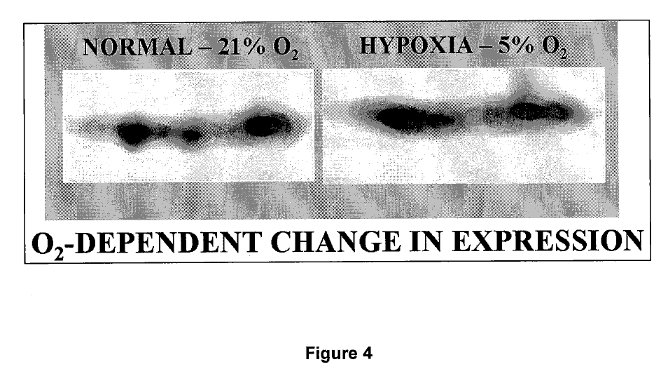

Abundant amounts of Hgb side-chains were found to be expressed in primary human RPE cells. Expression was confirmed by Western blotting (FIG. 1) and RT-PCR (FIG. 2). However, it was found that RPE cells rapidly downregulate Hgb expression in the tissue culture environment (FIG. 3). It was also found that hypoxia boosts Hgb expression (FIGS. 1 and 4) similar to VEGF and HIF1-α. It was therefore confirmed that Hgb is expressed in human RPE profusely and its production is increased by hypoxia.

Conclusions

Hgb is expressed in human RPE profusely and its production is increased by hypoxia. Without wishing to be bound by theory, a possible role for Hgb is to act as a reservoir and natural transporter of oxygen to the outer retina. However, other roles such as scavenging of NO, participation in oxidative damage and induction of RPE apoptosis and angiogenic signaling can also bear upon the clinical condition.

### Example 4

### Hemoglobin Expression in Human RPE

The present Example provides evidence that human retinal pigment epithelium (RPE) can produce and secrete Hgb into the Bruch's membrane. Hgb production by human RPE is dependent on the ambient oxygen and the presence of the neighboring tissues. Topographical and age-related differences in RPE-Hgb production suggest an explanation for several pathophysiological events of retinal disorders, including VEGF upregulation and iron toxicity.

Methods

The experimental protocols described herein were approved and monitored by the Institutional Review Board of the University of Louisville. Animal maintenance and experimentation was performed in accord with guidelines established by the Association for Research in Vision and Opthalmology, as well as with the Institutional Animal Care and Use Committee at the University of Louisville Medical School.

4-6 weeks old C57/B6 mice (Mus musculus; 20 eyes) and 8-weeks old brown Norway rats (Rattus norvegicus) were purchased from Jackson Laboratories (Bar Harbor, Me., U.S.A.). Freshly enucleated 10-12 week old domestic pig (Sus domestica) and bovine (>2 years old, Bos taurus) eyes were obtained from a local slaughterhouse.

Harvest of Primary RPE. Ten human donor eyes (age: 28-89) were obtained from Kentucky Lions Eye Bank (Louisville, Ky., U.S.A.) within 24 hours of death. Globes with previous history of any chorioretinal disease including diabetic retinopathy, AMD, myopia >6D, or inherited retinal degeneration were not included. Likewise, any donor eye that had undergone any major ocular surgery including laser photocoagulation was not included. Upon receipt, globes were cleaned of extraocular tissue and a small scleral incision was made 3 mm posterior to the limbus. Incision was then extended circumferentially. After the removal of the anterior segment, lens and ciliary body, suprachoroidal space was sealed applying cyanoacrylate glue circumferentially at ora serrata. Any residual vitreous was removed using sponges, scissors and a TEFLON®-coated spatula. Sensory retina was then gently peeled of exposing RPE cells which were washed three times with calcium-magnesium free Hank's balanced salt solution (HBSS) for 5 minutes. Loosened RPE cells were then gently peeled off and collected for further processing.

Topographic differences in Hgb expression were studied using 7.75 mm circular buttons of macular and peripheral eye wall buttons. Immediately after trepanation of the buttons, the suprachoroidal space was sealed using a cyanoacrylate glue to avoid any cellular contamination. The sensory retina was then gently peeled off exposing the underlying RPE. The exposed RPE layer was then washed three times with HBSS, and RPE cells were collected by gentle scraping. Cell count was determined with a COULTER COUNTER® (Coulter Scientific, Hialeah, Fla., U.S.A.). Viability (LIVE/DEAD® Cell Vitality Assay Kit, Invitrogen, Carlsbad, Calif., U.S.A.) and purity (RPE65, Chemicon, Temecula, Calif., U.S.A. and cytokeratin labeling kit, Sigma, St. Louis, Mo., U.S.A.) were determined as described previously (Tezel T H et al, Invest Opthalmol V is Sci.; 40(2): 467-476 (1999)). Only samples with >95% viability and >99% purity were used for further studies.

Effects of cell culturing on RPE Hgb expression were also studied for up to seven passages. For this reason, harvested RPE cells were incubated in a humidified atmosphere of 5% CO₂ and 95% air at 37° C. and maintained in Dulbecco's modified Eagle's medium (DMEM H16, Invitrogen, Carlsbad, Calif., U.S.A.) supplemented with 15% FBS, 100 IU/ml penicillin G, 100 μg/ml streptomycin, 5 μg/ml gentamicin, 2.5 μg/ml Amphotericin B and 1 ng/ml recombinant human basic fibroblast growth factor (bFGF, Invitrogen) to promote RPE cell growth. The medium was changed every other day and the cells observed daily. Cells became confluent in about 10 days and confluent cultures were passaged by trypsinization. First, third and seventh passage RPE cells were used to investigate the effects of Hgb expression in vitro.

Expression of Hgb in the RPE of C57/B6 mouse (20 eyes) and brown Norway rat (10 eyes) were also studied. For this purpose, eyes of the animals were enucleated while they were under deep anesthesia. Anterior segment was discarded and sensory retina was peeled off. Exposed RPE cells were washed three times with Hank's balanced salt solution and loosened RPE were scraped off from the Bruch's membrane using a TEFLON® spatula under a dissection microscope.

Protein Extraction. Harvested RPE cells were homogenized in sample buffer containing 7 M urea, 2.0 M thiourea, 65 mM CHAPS (3-[(3-Cholamidopropyl)dimethylammonio]-1-propanesulfonate hydrate), 58 mM DTT, 1.78% carrier ampholytes (pH 3-10) and 1× protease inhibitor cocktail (Sigma). The mixture was shaken at 4° C. for 1 hour and centrifuged at 15,000 g for 20 minutes at 4° C. The supernatant containing the soluble proteins was removed, and protein concentration in the supernatant was determined by spectrophotometry using a Bio-Rad Laboratories (Hercules, Calif., U.S.A.) protein microassay based on the method of Bradford by the Pierce BCA method (Bradford M M, Anal Biochem, 72: 248-254 (1976)). Samples were then stored at −80° C.

Two-Dimensional Gel Electrophoresis. 30 μg of protein from each sample was rehydrated overnight with immobilized pH gradient (IPG) strips (nonlinear pH 3-10), followed by isoelectric focusing for 90 minutes with maximal 2,000 V and 50 μA at room temperature using a two dimensional gel electrophoresis system (ZOOM® IPG Runner, Invitrogen). Second-dimension separation was done using 8×8 cm pre-cast polyacrylamide gels (XCELL SURELOCK™, Invitrogen). For this purpose, IPG strips were equilibrated twice for 10 minutes in a buffer containing 45 mM Tris base, pH 7.0, 6 M urea, 130 mM DTT, 30% glycerol, 1.6% SDS, and 0.002% bromophenol blue, followed by a third 10-minute incubation in the same buffer supplemented with 2.5% iodoacetamide instead of DTT was performed using precast 4-12% Bis-Tris gels (Invitrogen). Precision protein standards were run along with the sample at 200V using XCELL SURELOCK™ mini-cell electrophoresis system (Invitrogen).

The gel was removed after electrophoresis and fixed in 10% methanol and 7% acetic acid for 45 min followed by staining with SYPRO® Ruby (Molecular Probes, Eugene, Oreg., U.S.A.) overnight on a rocker at room temperature. The gel images after staining were captured using the PROXPRESS™ 2D Proteomic Imaging System (Perkin Elmer Life Sciences, Boston, Mass., U.S.A.) with excitation and emission wavelengths of 480 nm and 620 nm, respectively.

Image Analysis. Quantification and comparison of protein spots on gel images were done using Phoretix 2D Image Analysis Software (Nonlinear Dynamics, Newcastle upon Tyne, U.K.). This software was used to estimate Mᵣ and pI coordinates of the proteins, as well as to perform densitometric analysis and comparisons between different gels. Average mode of background subtraction was used to normalize the intensity prior to calculating spot volumes. Total spot volume was calculated to estimate the proportion of analyzed spot within the proteome. Using this software, identified protein spots were used to serve as a starting point for spot matching which was subsequently followed by additional spot matching based on pattern recognition. Accuracy of the protein spot matching was determined manually for each spot on each gel. Spot intensity was then compared for each spot identified and matched, and integrated intensity of each spot was determined. Mean and S.E.M. were calculated and a Mann-Whitney test was done on each spot to determine expression differences.

In-gel Tryptic Digestion. Samples were prepared using a modification of a technique described previously (Thongboonkerd V et al, J Biol Chem, 277(38): 34708-34716 (2002)). The stained gel slabs were extensively washed with 18 MΩ water, and spots were excised with a clean scalpel into a 1 mm cube. Ammonium bicarbonate (0.1 M) was then added to the gel pieces to twice the volume of the gel and incubated at room temperature for 15 min and acetonitrile was added to the gel pieces and incubated at room temperature for 15 min. The solvent was then removed and the gel pieces were dried at room temperature for 30 minutes; rehydrated with 20 μl of 20 mM dithiotreitol in 0.1 M NH₄HCO₃, and incubated at 56° C. for 45 minutes to reduce the protein. Thereafter, the tubes were cooled to room temperature; the dithiotreitol solution was removed and replaced with 20 μl of 55 mM iodoacetamide in 0.1 M NH₄HCO₃ and incubated at room temperature in the dark for 30 minutes. Next, the samples were washed again for 15 min in 0.2 ml of 50 mM NH₄HCO₃, before addition of 0.2 ml acetonitrile. At the end of 15 min incubation at room temperature, the solvent was removed and the gel pieces were dried and rehydrated with 5 ml of 20 ng/ml modified trypsin (Promega, Madison, Wis., U.S.A.) in 50 mM NH₄HCO₃. Next, rehydrated gel pieces were covered with 50 mM NH₄HCO₃ solution and incubated overnight at 37° C.

Sample Preparation for Mass-Spectrometry. A nitrocellulose solution was made by dissolving a nitrocellulose membrane in 1:1 acetone/isopropanol solvent. Alpha-cyano-4-hydroxycinnamic acid ([alpha]-CN) was washed with 50 μL of acetone and acetone phase was discarded. The [alpha]-CN was then dissolved in acetone to a concentration of 10 mg/mL, and the nitrocellulose and [alpha]-CN solutions were mixed to 1:4 ratio. 1 μL of this mixture was deposited onto the 96-well MALDI target plate. The sample was prepared for addition to the plate by adding 2 μL of sample to 2 μL of a solution of acetone washed [alpha]-CN dissolved in 0.1% trifluoroacetic acid and added to a 1:1 H₂O/acetonitrile to a final concentration of 10 mg/mL [alpha]-CN. One microliter of the sample mixture was then loaded onto each thin film. After the sample mixture was dried, 1.5 μL of 2% formic acid in deionized water was added to each spot. The formic solution was removed by gentle blotting. This washing step was performed twice. The samples were then dried at room temperature. Fragment size was determined by mass spectrometry.

Mass Spectrometry. Mass spectral data was obtained using a TOFSpec-2E mass spectrometer (Micromass, Manchester, U.K.). This instrument was equipped with a 337 nm N₂ laser at 20-35% power in the positive ion reflectron mode. Monoisotopic mass spectrum was be obtained by averaging 10 spectra each of which was the composite of 10 laser firings. The mass axis was calibrated using known peaks from tryptic autolysis.

Analysis of Peptide Sequence. Protein identification from tryptic fragment sizes was be made using the MASCOT™ search engine available from Matrix Science, Inc. (Boston, Mass., U.S.A.) based on the entire National Center for Biotechnology Information (NCBI) protein database using the assumption that peptides were monoisotopic, oxidized at methionine residues and carboxamidomethylated at cysteine residues. Up to one missed trypsin cleavage was allowed. A mass accuracy tolerance of a maximum of 150 ppm was used for matching tryptic peptides. Probability based MOWSE score defined as −10*log (P), where “p” is the probability that the observed match is a random event were determined. Score >70 was considered significant (p<0.05). Validity of the mass spectrometry data was ascertained by comparing the expected molecular weight and pI of the identified proteins with their position in the 2-D gel.

Western Blotting. Extracted RPE proteins were denatured at 70° C. for 10 minutes and equal amounts (20 μg in 20 μl/lane) of total proteins were loaded on NUPAGE® NOVEX® 4-12% gels (Invitrogen). Gels were then transferred onto nitrocellulose membranes (Invitrogen) at 30 V for 1 h in transfer buffer (25 mM Tris base, 192 mM glycine, 20% (v/v) methanol, 0.1% (w/v) SDS, pH 8.3). After blocking the membranes for 1 h in Tris-buffered saline (50 mM Tris, pH 7.5, 150 mM NaCl) containing 5% (w/v) bovine serum albumin, blots were incubated overnight at 4° C. with horseradish peroxidase conjugated anti-human Hgb antibody (Bethyl Laboratories, Montgomery, Tex., U.S.A.) diluted 1:2000 in PBS. In control samples Western blots were developed with the ECL chemiluminescence (Amersham Biosciences, Piscataway, N.J., U.S.A.) and exposed to BIOMAX® XAR film (Eastman Kodak Company, Rochester, N.Y., U.S.A.). Hgb expression further characterized by determining the expression of different Hgb side chains with anti-human [symbol] ,α2, β, and γ globulin antibodies. The presence of other erythrocyte markers was also probed with antihuman CD233 (Aviva Systems Biology, San Diego, Calif., U.S.A.) and antibodies against blood group H and AB antigens (Abcam, Cambridge, U.K.) at 1:1000 dilution.

RNA Preparation and Reverse Transcription Polymerase Chain Reaction (RT-PCR). Expression of Hgb α and β side-chain mRNAs in human and rat RPE were determined by RT-PCR as described previously (Baumforth K R et al, Mol Pathol, 52(1):1-10 (1999)). In brief, human RPE mRNA was extracted from four fresh donor (ages: 48-65; death-to-harvest time <24 hours) eyes, as well as 20 C57/B6 and 10 brown Norway rat eyes by using TRIZOL® (Invitrogen). Harvested RPE washed three times with Hank's balanced cell solution before adding 2 ml of TRIZOL® on the cell pellet. It was then mixed with 0.4 ml of chloroform and centrifuged at 14,000 rpm for 15 minutes at 4° C. Pelleted RNA was then washed with isopropanol for 30 minutes and 75% ethanol for 10 minutes at 4° C. RNA was then dissolved in 30 μl of RNase-free water. Any possible DNA contamination was removed by incubation with amplification grade DNase I (Invitrogen) and confirmed by PCR analysis of total RNA samples prior to reverse transcription. After isolation, the integrity and size distribution of the RNA samples was checked by agarose gel electrophoresis. The yield and purity of total RNA was determined by measuring the absorbance (260/280 nm) of ethanol precipitated aliquots of the samples. Oligo-dT primed first strand cDNA was synthesized with using SUPERSCRIPT® 3 First Strand Synthesis System for RT-PCR (Invitrogen) using 1 g of total RNA per reaction. PCR amplification of cDNAs was performed using a Stratagene Mx3000P (Stratagene, La Jolla, Calif., U.S.A.) system using Taq DNA polymerase (Promega) and primers corresponding to conserved sequences of known human, mouse and rat Hgb side-chain genes. All the primers were designed to cross exon-intron borders, to exclude the amplification of genomic templates. Primer sets of β-actin were used as internal controls. Denaturation was started initially at 95° C. for 4 minutes, followed by 40 amplification cycles of 95° C. for 15 seconds, at 60° C. for 20 seconds, and at 72° C. for 20 seconds, for primer annealing, and 7 minutes at 72° C. for primer extension. Control reactions for each primer pair were also performed with the transcription control reaction without reverse transcriptase, and with water instead of cDNA template. PCR products were then separated on 2% agarose gels and bands were visualized with ethidium bromide.

Immunohistochemistry. Localization of Hgb in the outer retina was examined with light and electron microscopy. For light microscopy full-thickness human and mouse eye wall samples were fixed overnight with 4% paraformaldehyde at 4° C.; washed three times with PBS and embedded in paraffin. 5 μm-thick sections were cut; deparaffinized in xylene and rehydrated in graded alcohol. Samples were then immunostained with a goat anti-human Hgb antibody (1 mg/ml, Bethyl Laboratories) in 1:500 dilution for 2 hours at room temperature. After washing three times with PBS, sections were incubated with Cy3-conjugated anti-goat IgG in 1:10 dilution (Sigma) for 1 hour at room temperature. Visualization of the Hgb was achieved by viewing the sample with 60× (1.4 NA) or 100× (1.3 NA) oil immersion objectives under a fluorescence microscope equipped with appropriate emission (540-552 nm) and excitation (590 nm) filters (Axiovision, Carl Zeiss, Thornwood, N.Y., U.S.A.). Images of 1280×1024 pixels were captured with a digital camera.

For immunoelectron microscopy tissue samples were fixed with 4% paraformaldehyde/0.5% glutaraldehyde in 0.1 M sodium cacodylate, pH 7.2 at 4° C. overnight. Tissue samples were then rinsed with PBS and dehydrated in graded series of up to 100% ethanol. Tissue samples were then infiltrated with LR gold resin and placed in gelatin capsules for polymerization under ultraviolet light for 18 hours. Ultrathin sections were immunostained at room temperature for 2 hours initially with mouse monoclonal anti-Hgb antibody (Abcam) at 1:100 dilution followed by 10 nm-gold particle-conjugated anti-mouse IgG (Sigma) at 1:500 dilution. Following immunostaining sections were counterstained with 1% uranyl acetate and lead citrate. For negative controls primary antibody was substituted with non-immunized fetal bovine serum (1:500). Samples were examined with a JEOL 200CX electron microscope (Tokyo, Japan).

Hgb Release from Human RPE. Hgb released into the extracellular space was determined using an enzyme-linked immunosorbent assay (ELISA) for human Hgb (Bethyl Laboratories, Montgomery, Tex., U.S.A.). For this purpose, 7 pairs of freshly enucleated (within 24 hours of death) human globes were obtained from the eye bank (57-70 years old). Anterior segment, vitreous and retina were removed exposing the RPE cells. Cyanoacrylate glue was used to seal the suprachoroidal space. Eyecups were then washed gently three times and incubated in a humidified atmosphere of 5% CO₂ and 95% air at 37° C. and maintained in Dulbecco's modified Eagle's medium (DMEM H16, Gibco) supplemented with 15% FBS, 100 IU/ml penicillin G, 100 μg/ml streptomycin, 5 μg/ml gentamicin, 2.5 □g/ml Amphotericin B for 16 hours. RPE of the fellow eyes were gently scraped from the Bruch's membrane after three washings with calcium-magnesium free Hank's balanced salt solution. Care was taken not to disrupt the integrity of the underlying Bruch's membrane. Collected cells were washed once with culture medium and cell viability was determined with LIVE/DEAD® Cell Vitality Assay Kit (Invitrogen). RPE cells suspensions and fellow eyecups devoid of RPE were also incubated with the culture medium for 16 hours. At the end of the incubation period, culture media were collected from all experimental groups; centrifuged and the amount of Hgb in the medium was determined with ELISA. For this purpose, microtiter plates were coated with sheep anti-human Hgb antibody (10 μg/mL, Bethyl Laboratories) and incubated for 1 hour at room temperature. The supernatant fluid was removed and the plates were washed three times with 50 mM Tris, 0.14 M NaCl, 0.05% Tween 20 pH 8.0 and blocked with 200 μl/well 50 mM Tris, 0.14 M NaCl, 1% BSA pH 8.0 for 30 minutes at room temperature. The plates were washed three times and 3 μg of RPE protein in 50 mM Tris, 0.14 M NaCl, 1% BSA, 0.05% Tween pH 8.0 was added. After 30 minutes of incubation at room temperature plates were washed five times and incubated for 60 minutes at room temperature with 100 μl of horseradish peroxidase conjugated sheep anti-human Hgb antibody (1 mg/ml) diluted 1:50,000 followed by five serial washings. 100 μl of chromogenic substrate 3,3′,5,5′,-tetramethyl-benzidine (Bethyl Laboratories) in 0.05 M citric acid buffer at pH 4.0, containing 5 μl hydrogen peroxide (30% wt/vol)/20 ml, was added to all wells. The chromophore development was terminated after 30 min at 25° C. with 2 NH₂ SO₄. The plates were read at a wavelength of 450 nm using VERSAMAX™ microplate reader (Molecular Devices, Sunnyvale, Calif., U.S.A.). Amount of Hgb was calculated using the normalization curve obtained from wells containing dilutions of human Hgb ranging from 16 ng/ml to 1 mg/ml. Obtained data was corrected to live RPE cell count to estimate the Hgb content of each RPE cell.

In a similar manner, the proportion of Hgb F within RPE intracellular Hgb pool was calculated for each sample using an ELISA assay specific for Hgb F. This time affinity-purified sheep anti-human Hgb F antibodies (Bethyl Laboratories) were used to quantify Hgb F.

Statistical Analysis. For all experiments with quantitative results, the data was expressed as the mean±standard deviation. Data from three identical experiments carried out independently. Data from all experiments were pooled and expressed as mean±standard deviation. Differences between RPE cell populations were analyzed in pairs by Dunn's multiple comparison test (Daniel W. Biostatistics. A Foundation for Analysis in the Health Sciences. 6th edition ed. 1995, New York, N.Y.: John Wiley & Sons. 273-352). A confidence level of p<0.05 was considered to be statistically significant.

Results

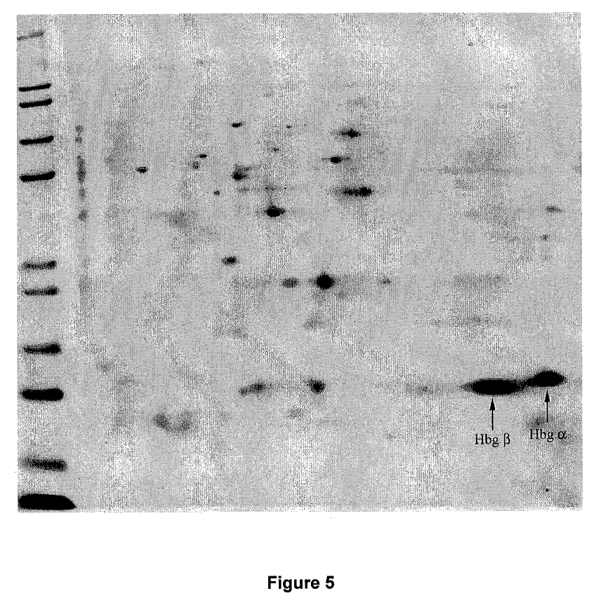

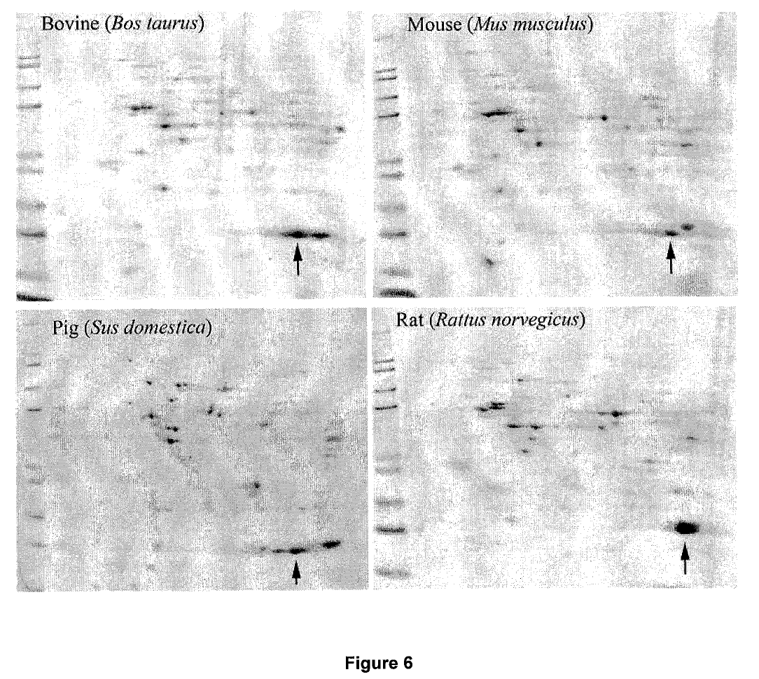

Presence of Hgb within RPE Proteome. Hgb side chains (α and β) were clearly detectable in RPE proteome of all studied human eyes regardless of gender and age. Components of human Hgb were clustered at around 15 kDa and a pI zone of 6.5-9.0 (FIG. 5). Among these spots, mass spectrometry was able to identify Hgb α and α2 chains (molecular weight: 15.2 kDa; pI; 8.72), β chain (molecular weight: 15.9 kDa; pI; 6.7), as well as third (Silva M M et al, J Biol Chem, 267(24):17248-17256 (1992)) and recently identified quaternary structures of Hgb a (molecular weight: 15.9 kDa; pI; 6.8) (Safo M K and Abraham D J, Biochemistry, 44(23):8347-8359 (2005)) (FIG. 5). Hgb expression of human RPE cells declined upon culturing but persisted up to seven passages (FIG. 3). Hgb side chains were also present in the proteomes RPE of other studied mammals, including rat, mouse, pig and bovine (FIG. 6).

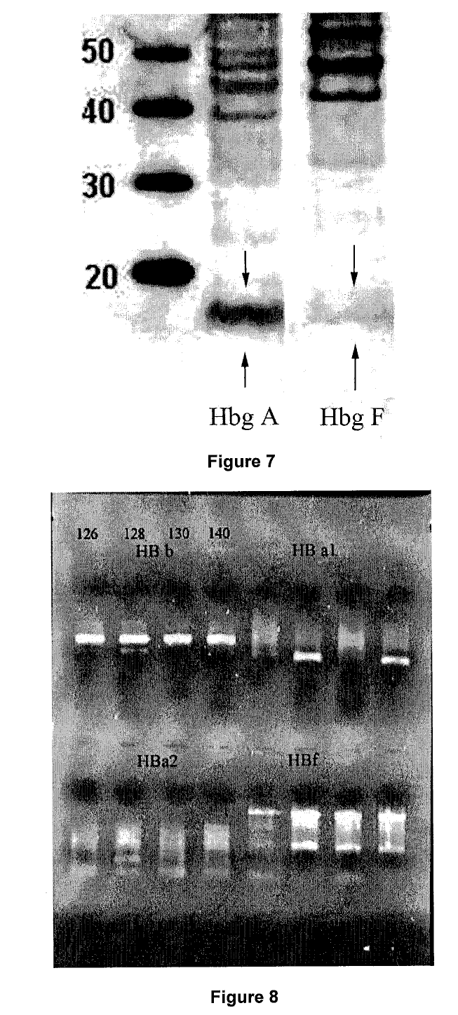

Presence of Hgb within human RPE was also confirmed with Western blotting (FIG. 7). Western blotting demonstrated the presence of Hgb [symbol] ,α2, β, and γ globulin side chains within RPE (FIG. 7). However, it failed to show the presence of erythrocyte-specific proteins, such as CD233, and blood group H and AB antigens within human RPE, indicating that the source of Hgb was not phagocytosis.

Expression of Hgb by RPE. RT-PCR from primary human RPE demonstrated the expression Hgb α1, α2, γ and β chains by RPE cells (FIG. 8). Similarly, mouse and rat RPE were also shown to express Hgb (FIG. 8).

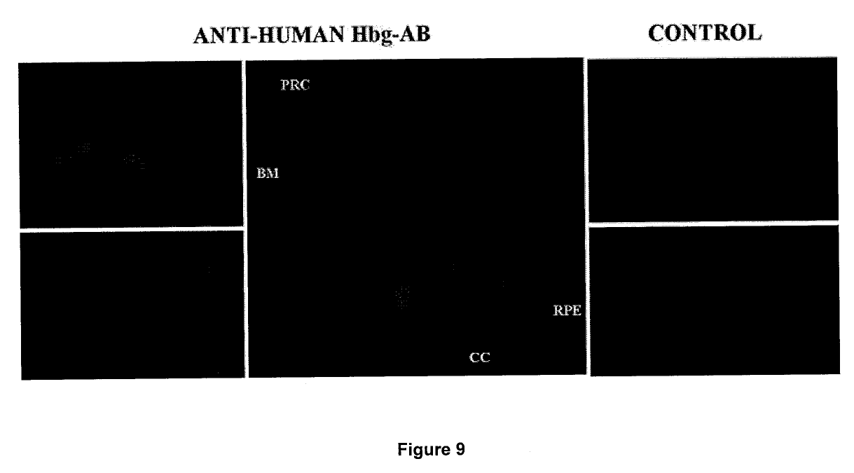

Immunohistochemistry. Light microscopy confirmed the presence of Hgb within human RPE as small granular deposits located predominantly close to the basal membrane (FIG. 9). Some of the signal appeared to be extracellular originating from the inner layers of Bruch's membrane. Granular deposits of Hgb were also detected on erythrocytes membrane within choriocapillaris. A more diffuse staining pattern was also observed in the rat and mouse eyes. Hgb signal was less intense compared with human eyes.

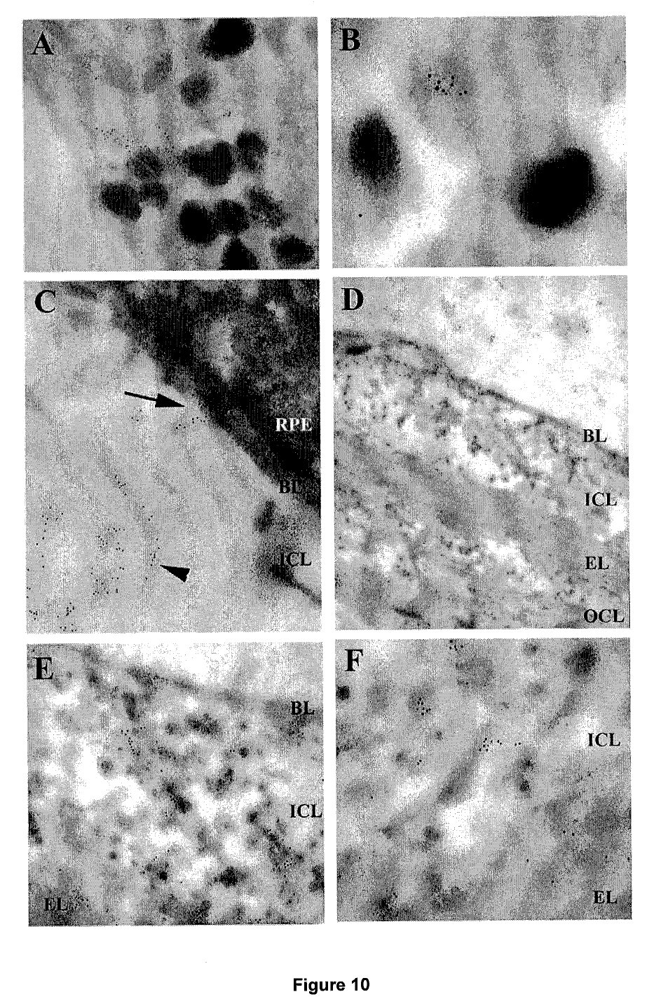

Immunoelectron microscopy confirmed the presence of Hgb within RPE cells (FIGS. 10A-9F). Immunogold particles indicative of Hgb immunoreactivity were present within RPE cytosol (FIG. 10A), mainly in multiple, round, intermediate-to-high density cytoplasmic granules (FIG. 10B). Hgb was exocytosed from the basal membrane of RPE (FIG. 10C).

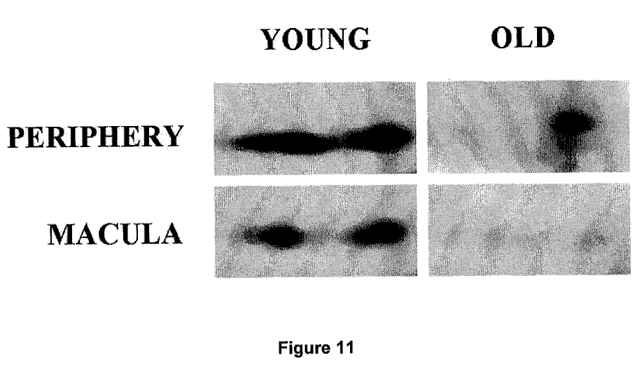

The data presented in Examples 1-4 demonstrate the presence of Hgb both in the RPE basolateral membrane as well as all along the inner aspect of the Bruch's membrane in human and mouse. The data also show age-related decline in the amount of Hgb in the Bruch's membrane especially in the macula (FIG. 11), which indicates a decline in oxygen transport through the Bruch's membrane in the macula by aging. The decrease in Hbg content of the aged macula indicates that the oxygen transport mechanism can be disrupted by decreased production of Hbg in the outer retina, which without wishing to be bound by theory, may be a reason for the development of hypoxia-induced cellular damage and pathological events leading to AMD. Again, without wishing to be bound by theory, these observations can explain the relative hypoxia which plays a role in cellular death and initiation of angiogenic response in age-related macular degeneration and therefore diagnostic methods for measuring hemoglobin and modified hemoglobin in the subject as disclosed herein can be useful for diagnosing a retinal disorder and/or the risk of developing the disorder in a subject. Further, therapeutic methods disclosed herein can be used to treat retinal disorders by decreasing hypoxia in the retinal tissue resulting from insufficient functional hemoglobin in the retinal tissue.

### REFERENCES

Ahmed N et al, Diabetologia, 48(8):1590-603 (2005).
Amiconi G et al, Adv Exp Med Biol, 307:185-90 (1991).
Baumforth K R et al, Mol Pathol, 52(1):1-10 (1999).
Beyerbach A et al, Carcinogenesis, 27(8):1600-6 (2006).
Bradford M M, Anal Biochem, 72: 248-254 (1976).
Brannon-Peppas L & Blanchette J O, Adv Drug Deliv Rev 56(11): 1649-59 (2004).
Broillet M C, Cell Mol Life Sci, 55(8-9):1036-42 (1999).
Bry L et al, Clin Chem, 47(2):153-63 (2001).
Bunn H F, Haematologia (Budap), 17(2):179-86 (1984).
Cerami A, Prog Clin Biol Res, 195:79-90 (1985).
Chen et al, Proc. Natl. Acad. Sci., USA 91: 3054-3057 (1994).
Culture Of Animal Cells, R. I. Freshney, Alan R. Liss, Inc. (1987).
Daniel W. Biostatistics. A Foundation for Analysis in the Health Sciences. 6th edition ed. 1995, New York, N.Y.: John Wiley & Sons. 273-352.
Dev et al, Cancer Treat. Rev. 20:105-115 (1994).
DNA Cloning, Volumes I and II, Glover, ed. (1985).
FercherAF, J. Biomed. Opt., 1(2) 157-173 (1996).
Fraser J et al, Proc Natl Acad Sci USA (2006).
Gene Transfer Vectors For Mammalian Cells, J. H. Miller and M. P. Calos, eds., Cold Spring Harbor Laboratory (1987).
Handbook Of Experimental Immunology, Volumes I-IV, D. M. Weir and C. C. Blackwell, eds. (1986).
Harding J J, Adv Protein Chem, 37:247-334 (1985).
Huang D et al., Science 254: 1178-1181 (1991).
Immobilized Cells And Enzymes, IRL Press (1986).
Immunochemical Methods In Cell And Molecular Biology (Mayer and Walker, eds., Academic Press, London (1987).
Kasten-Jolly J and Abraham E C, Biochim Biophys Acta, 27; 913(1):89-91 (1987).
Kolkman et al, Lasers Med Sci, 21(3):134-9 (2006).
LaVail et al, Invest. Opthalmol. Vis. Sci. 39:592-602 (1998).
Liu L et al, Proc Natl Acad Sci USA (1999).
Marzocchi B et al, Pediatr Res. October; 58(4):660-5 (2005).
Methods In Enzymology, Vols. 154 and 155, Wu et al., eds., Academic Press Inc., N.Y.
Miksik I and Deyl Z, J Chromatogr B Biomed Sci Appl. 699 (1-2):311-45 (1997).
Molecular Cloning A Laboratory Manual (1989), 2nd Ed., ed. by Sambrook, Fritsch and Maniatis, eds., Cold Spring Harbor Laboratory Press, Chapters 16 and 17.
Nagai R et al, Ann N Y Acad. Sci., 1043:146-50 (2005).
Newton D A et al, J Biol Chem (2006).
Niketic V et al, Biochem Biophys Res Commun. October 20; 239(2):435-8 (1997).
Nucleic Acid Hybridization, D. Hames & S. J. Higgins, eds. (1984).
Oligonucleotide Synthesis, M. J. Gait, ed. (1984).
Ostojić J et al, Invest Opthalmol V is Sci (2006).
Perbal, A Practical Guide To Molecular Cloning; See Methods In Enzymology (Academic Press, Inc., N.Y.) (1984).
Quteba E et al, Proc Natl Acad Sci USA 2006 Sep. 5; 103(36):13480-4.
Remmington's Pharmaceutical Sciences, Meade Publishing Co., Easton, Pa.
Report of the Retinal and Choroidal Panel: Vision Research—A National Plan 1983-1987, vol. 2, part 1, summary page 2.
Rodkey F L et al, Clinical Chemistry, 25:1388-1393 (1979).
Safo M K and Abraham D J, Biochemistry, 44(23):8347-8359 (2005).
Schillinger M et al, Circulation (2003).
Schweitzer D et al, Klin Monatsbl Augenheilkd, 222(5):396-408 (2005).
Silva M M et al, J Biol Chem, 267(24):17248-17256 (1992).
S\acute{ø}rensen M et al, Environ Health Perspect (2003).
Sorg B S et al, J Biomed Opt, 10(4):44004 (2005).
Srinivasan S et al, Acad Radiol, 13(2):195-202 (2006).
Tezel T H et al, Invest Opthalmol V is Sci.; 40(2): 467-476 (1999).
Thongboonkerd V et al, J Biol Chem, 277(38): 34708-34716 (2002).
Transcription and Translation, B. D. Hames & S. J. Higgins, eds. (1984).
U.S. Pat. No. 4,268,270.
U.S. Pat. No. 4,683,195.
U.S. Pat. No. 5,091,513.
U.S. Pat. No. 5,279,833.
U.S. Pat. No. 5,286,634.
U.S. Pat. No. 5,328,470.
U.S. Pat. No. 5,399,346.
U.S. Pat. No. 5,632,984.
U.S. Pat. No. 5,641,484.
U.S. Pat. No. 5,643,567.
U.S. Pat. No. 5,646,008.
U.S. Pat. No. 5,651,964.
U.S. Pat. No. 6,046,223.
U.S. Pat. No. 6,169,068.
U.S. Pat. No. 6,458,587.
U.S. Pat. No. 6,489,162.
U.S. Pat. No. 6,491,907.
U.S. Pat. No. 6,548,286.
U.S. Pat. No. 7,018,826.
U.S. Pat. No. 7,022,319.
U.S. Pat. No. 7,052,881.
U.S. Pat. No. 7,105,346.
Vijayanathan V et al, Biochemistry, 41(48):14085-94 (2002).
Vrouenraets M B et al, Anticancer Research, 23:505-522 (2003).
Wang et al, J Biomed Opt, 11(2):024015 (2006).
Webb, R H et al, Applied Optics, 26: 1492-1499 (1987).
Winterhalter K H, Prog Clin Biol Res.; 195:109-22 (1985).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for diagnosing a retinal disorder or a risk of developing the retinal disorder in a subject, comprising:
(a) providing a biological sample from the subject;
(b) determining an amount of modified hemoglobin in the sample; and
(c) comparing the amount of modified hemoglobin to a control level, wherein the subject is diagnosed as having the retinal disorder or being at risk for developing the retinal disorder if there is a measurable difference in the amount of modified hemoglobin in the sample as compared to the control level.

2. The method of claim 1, wherein the retinal disorder is a retinal disorder selected from the group consisting of age-related macular degenerations, diabetic retinopathies, retinopathy of prematurity (ROP), ischemic vasculopathies, inherited retinal dystrophies, retinal detachment, aberrant angiogenesis, and glaucoma.

3. The method of claim 1, wherein the retinal disorder produces in the subject one or more physiological abnormalities selected from the group consisting of choroidal neovascularization (CNV), retinal angiomatous proliferation (RAP), intraretinal microvascular abnormalities, pre-retinal neovascularization, choroidal angiogenesis, and choroidal vasculopathy.

4. The method of claim 1, wherein the biological sample comprises blood, plasma, serum, or ocular fluids.

5. The method of claim 1, wherein the modified hemoglobin comprises oxidized hemoglobin, s-nitrosylated hemoglobin, acetylated hemoglobin, glycated hemoglobin, or combinations thereof.

6. The method of claim 1, wherein the subject is human.

7. A method for diagnosing a retinal disorder or the risk of developing the retinal disorder in a subject, comprising:
(a) determining an amount of hemoglobin, modified hemoglobin, or both present in retinal tissue of the subject; and
(b) comparing the amount of hemoglobin, modified hemoglobin, or both to a control level, wherein the subject is diagnosed as having the retinal disorder or being at risk for developing the retinal disorder if there is a measurable difference in the amount of hemoglobin, modified hemoglobin, or both in the retinal tissue as compared to the control level.

8. The method of claim 7, wherein the retinal disorder is a retinal disorder selected from the group consisting of age-related macular degenerations, diabetic retinopathies, retinopathy of prematurity (ROP), ischemic vasculopathies, inherited retinal dystrophies, retinal detachment, aberrant angiogenesis, and glaucoma.

9. The method of claim 7, wherein the retinal disorder produces in the subject one or more physiological abnormalities selected from the group consisting of choroidal neovascularization (CNV), retinal angiomatous proliferation (RAP), intraretinal microvascular abnormalities, pre-retinal neovascularization, choroidal angiogenesis, and choroidal vasculopathy.

10. The method of claim 7, wherein determining the amount of hemoglobin, modified hemoglobin, or both comprises non-invasive imaging of the retinal tissue to measure the amount of hemoglobin, modified hemoglobin, or both present in the retinal tissue.

11. The method of claim 7, wherein the modified hemoglobin comprises oxidized hemoglobin, s-nitrosylated hemoglobin, acetylated hemoglobin, glycated hemoglobin, or combinations thereof.

12. The method of claim 7, wherein the retinal tissue comprises retinal pigment epithelial (RPE) cells, Bruch's membrane, choroid, or combinations thereof.

13. The method of claim 7, wherein the subject is human.

14. A method for determining whether to initiate or continue prophylaxis or treatment of a retinal disorder in a subject, comprising:
(a) providing a series of biological samples over a time period from the subject;
(b) analyzing the series of biological samples to determine an amount of modified hemoglobin in each of the biological samples; and
(c) comparing any measurable change in the amounts of modified hemoglobin in each of the biological samples to determine whether to initiate or continue the prophylaxis or therapy of the retinal disease.

15. The method of claim 14, wherein the retinal disorder is a retinal disorder selected from the group consisting of age-related macular degenerations, diabetic retinopathies, retinopathy of prematurity (ROP), ischemic vasculopathies, inherited retinal dystrophies, retinal detachment, aberrant angiogenesis, and glaucoma.

16. The method of claim 14, wherein the retinal disorder produces in the subject one or more physiological abnormalities selected from the group consisting of choroidal neovascularization (CNV), retinal angiomatous proliferation (RAP), intraretinal microvascular abnormalities, pre-retinal neovascularization, choroidal angiogenesis, and choroidal vasculopathy.

17. The method of claim 14, wherein the biological samples are each independently selected from the group consisting of blood, plasma, serum, or ocular fluids.

18. The method of claim 14, wherein the series of biological samples comprises a first biological sample collected prior to initiation of the prophylaxis or treatment for the retinal disorder and a second biological sample collected after initiation of the prophylaxis or treatment.

19. The method of claim 14, wherein the series of biological samples comprises a first biological sample collected prior to onset of the retinal disorder and a second biological sample collected after onset of the retinal disorder.

20. The method of claim 14, wherein the modified hemoglobin comprises oxidized hemoglobin, s-nitrosylated hemoglobin, acetylated hemoglobin, glycated hemoglobin, or combinations thereof.

21. The method of claim 14, wherein the subject is human.

22. A method for determining whether to initiate or continue prophylaxis or treatment of a retinal disorder in a subject, comprising:
(a) determining a series of amounts of hemoglobin, modified hemoglobin, or both present in retinal tissue of the subject over a time period; and
(b) comparing any measurable change in the amounts of hemoglobin, modified hemoglobin, or both present in the retinal tissue of the subject over the time period to determine whether to initiate or continue the prophylaxis or therapy of the retinal disease.

23. The method of claim 22, wherein the retinal disorder is a retinal disorder selected from the group consisting of age-related macular degenerations, diabetic retinopathies, retinopathy of prematurity (ROP), ischemic vasculopathies, inherited retinal dystrophies, retinal detachment, aberrant angiogenesis, and glaucoma.

24. The method of claim 22, wherein the retinal disorder produces in the subject one or more physiological abnormalities selected from the group consisting of choroidal neovascularization (CNV), retinal angiomatous proliferation (RAP), intraretinal microvascular abnormalities, pre-retinal neovascularization, choroidal angiogenesis, and choroidal vasculopathy.

25. The method of claim 22, wherein determining the amounts of hemoglobin, modified hemoglobin, or both comprises non-invasive imaging of the retinal tissue to measure the amounts of hemoglobin, modified hemoglobin, or both present in the retinal tissue over the time period.

26. The method of claim 22, wherein the time period begins prior to initiation of the prophylaxis or treatment for the retinal disorder and ends after initiation of the prophylaxis or treatment.

27. The method of claim 22, wherein the time period begins prior to onset of the retinal disorder and ends after onset of the retinal disorder.

28. The method of claim 22, wherein the modified hemoglobin comprises oxidized hemoglobin, s-nitrosylated hemoglobin, acetylated hemoglobin, glycated hemoglobin, or combinations thereof.

29. The method of claim 22, wherein the retinal tissue comprises retinal pigment epithelial (RPE) cells, Bruch's membrane, choroid, or combinations thereof.

30. The method of claim 22, wherein the subject is human.

31. A method of treating a retinal disorder in a subject, comprising decreasing hypoxia in retinal tissue of the subject by increasing an amount of hemoglobin, increasing an activity of hemoglobin, decreasing an amount of modified hemoglobin, decreasing an activity of modified hemoglobin, or combinations thereof in the retinal tissue of the subject.

32. The method of claim 31, wherein the retinal disorder is a retinal disorder selected from the group consisting of age-related macular degenerations, diabetic retinopathies, retinopathy of prematurity (ROP), ischemic vasculopathies, inherited retinal dystrophies, retinal detachment, aberrant angiogenesis, and glaucoma.

33. The method of claim 31, wherein the retinal disorder produces in the subject one or more physiological abnormalities selected from the group consisting of choroidal neovascularization (CNV), retinal angiomatous proliferation (RAP), intraretinal microvascular abnormalities, pre-retinal neovascularization, choroidal angiogenesis, and choroidal vasculopathy.

34. The method of claim 31, wherein the retinal tissue comprises retinal pigment epithelial (RPE) cells, Bruch's membrane, choroid, or combinations thereof.

35. The method of claim 31, wherein the modified hemoglobin comprises oxidized hemoglobin, s-nitrosylated hemoglobin, acetylated hemoglobin, glycated hemoglobin, or combinations thereof.

36. The method of claim 31, wherein increasing the amount of hemoglobin in the retinal tissue comprises administering a therapeutically effective amount of erythropoietin to the subject.

37. The method of claim 31, wherein decreasing the concentration of modified hemoglobin in the retinal tissue comprises removing retinal tissue comprising the modified hemoglobin.

38. The method of claim 31, wherein decreasing the activity of modified hemoglobin comprises administering a pharmaceutical composition comprising an anti-CD36 molecule to the subject.

39. The method of claim 37, wherein the anti-CD36 molecule is an antibody or an aptamer.

40. The method of claim 31, wherein the subject is human.
